(19) United States
(12) Patent Application Publication
(10) Pub. No.: US 2007/0295607 A1
(43) Pub. Date: Dec. 27, 2007

(54) RESIN COMPOSITION FOR INTERLAYER INSULATING LAYER OF MULTI-LAYER PRINTED WIRING BOARD

(75) Inventor: Kenji KAWAI, Kawasaki-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: AJINOMOTO CO. INC, Tokyo (JP)

(21) Appl. No.: 11/564,403

(22) Filed: Nov. 29, 2006

(30) Foreign Application Priority Data
Nov. 29, 2005 (JP) 344721/2005
Feb. 22, 2006 (JP) 045864/2006

(51) Int. Cl. C25D 5/56; C07D 303/00

(52) U.S. Cl. 205/164; 523/400; 525/109

(57) ABSTRACT

Epoxy resin compositions which comprise (A) an epoxy resin having two or more epoxy groups in one molecule, (B) a phenolic curing agent having an average content ratio of hydroxyl group in one molecule (P) of 0<P<1, and (C) a polyvinyl acetal resin are useful for an insulating layer of a multi-layer printed wiring board, which can afford an insulating layer (interlayer insulating layer) whose roughened surface shows a high cohesive force to a plated conductor, even though the roughness of the roughened surface after a roughening treatment is comparatively small

### CROSS REFERENCES TO RELATED APPLICATIONS

This application claims priority to Japanese Patent Application No. 2005-344721 filed on Nov. 29, 2005, and Japanese Patent Application No. 2006-45864, filed on Feb. 22, 2006, both of which are incorporated herein by reference in their entireties.

### BACKGROUND OF THE INVENTION

1. Field of the Invention

The present invention relates to epoxy resin compositions which are useful for forming an insulating layer of a multi-layer printed wiring board.

2. Discussion of the Background

With the advancement of downscaling and high performance of electronics in recent years, plural buildup layers are employed for multi-layer printed wiring boards, and ultrafine and high density wiring has been increasingly achieved. As conductor formation methods suitable for forming a high density, ultrafine wiring, there are known an additive method comprising roughening the surface of an insulating layer, and forming a conductive layer by electroless plating, and a semi-additive method comprising forming a conductive layer by electroless plating and electroplating. In these methods, cohesion between an insulating layer and a plated conductive layer (plated copper) is mainly ensured by the surface of the insulating layer processed by a roughening treatment, i.e., irregularities on the surface of the resin layer. By the presence of the irregularities on the surface of the insulating layer, an anchor effect is obtained for the plating layer. Accordingly, for an enhanced cohesive force, the level of irregularities (roughness) of the surface of an insulating layer may be increased.

However, for a still higher density of wiring, the roughness of the surface of the insulating layer is preferably made small. To be specific, when the surface of the insulating layer has high roughness when, after formation of a conductive layer by electroless plating or electroplating, wiring formation is completed by removing the plating layer of a thin film by flash etching, flash etching for a long-time is necessary to remove smear that invaded into the concave part, and a prolonged flash etching increases the risk of damaging or breaking the ultrafine wiring. To form a highly reliable high density wiring, therefore, the surface of an insulating layer is required to be superior in cohesion with the plated conductor even when the roughness after a roughening treatment is small. However, an insulating material capable of forming a roughened surface showing such property has not been developed.

JP-A-2005-154727 and US 2005008868 A1 disclose that an epoxy resin composition comprising two particular kinds of epoxy resins, a phenolic curing agent, a particular thermoplastic resin (polyvinyl acetal, phenoxy resin etc.) and an inorganic filler in a particular ratio, which is used for an insulating layer of a multi-layer printed wiring board, shows a low coefficient of thermal expansion and a superior peel strength of a conductive layer formed by plating.

JP-A-2003-286390 discloses that an epoxy resin composition comprising particular amounts of an epoxy resin, a polyvalent hydroxy resin curing agent, a bisphenol A or F phenoxy resin, a particular rubber component and a curing accelerator, which is used as an insulating layer of a multi-layer printed wiring board and the like, is superior in heat resistance, mechanical strength, film supportability, and the like.

JP-A-2003-286391 discloses that an epoxy resin composition comprising particular amounts of an epoxy resin, a particular phenolbiphenylaralkyl resin curing agent, a particular phenoxy resin, a particular rubber component and a curing accelerator, which is used as an insulating layer of a multi-layer printed wiring board and the like, is superior in heat resistance, mechanical strength, film supportability, and the like.

However, even in the patent references disclosing various such resin compositions, a resin composition which has a low roughness and high peel strength when used as an insulating layer has not been disclosed, and such problem to be solved is not indicated.

### SUMMARY OF THE INVENTION

Accordingly, it is one object of the present invention to provide novel epoxy resin compositions which are useful as insulating layers of multi-layer printed wiring boards.

It is another object of the present invention to provide novel epoxy resin compositions which are useful as insulating layers of multi-layer printed wiring boards, which can afford an insulating layer whose roughened surface shows a high cohesive force to a plated conductor, even though the roughness of the roughened surface after a roughening treatment is comparatively small.

These and other objects, which will become apparent during the following detailed description, have been achieved by the inventors' discovery that, as regards an epoxy resin composition containing an epoxy resin, polyvinyl acetal, and a particular phenolic curing agent, when the epoxy resin is cured and the obtained cured product is roughened, the resulting roughened surface affords close adhesion to a plated conductor with a high cohesive force, even if the roughness is comparatively small, which resulted in the completion of the present invention.

Accordingly, the present invention provides the following:

(1) An epoxy resin composition comprising:

(A) an epoxy resin having two or more epoxy groups in one molecule;

(B) a phenolic curing agent having an average content ratio of hydroxyl group in one molecule (P) of 0<P<1; and

(C) a polyvinyl acetal resin,

wherein the average content ratio of hydroxyl group in one molecule (P) is an average value of H/B, where H is the total number of hydroxyl groups and B is the total number of benzene rings.

(2) The epoxy resin composition of the above-mentioned (1), wherein the phenolic curing agent (B) is a phenolic curing agent represented by formula (1) or (2): 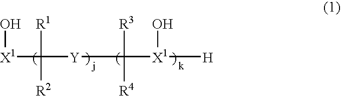
wherein R¹ to R⁴ are each independently hydrogen atom or an alkyl group; X⁻¹ is a benzene ring, hydroxyl benzene ring, naphthalene ring or hydroxyl naphthalene ring each of which may be substituted with an alkyl group; Y is a benzene ring, hydroxyl benzene ring or a biphenyl ring each of which may be substituted with an alkyl group; and j and k are each independently number of 1 to 15 as average value, 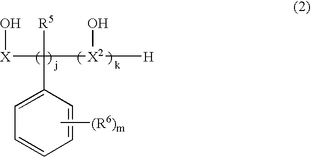
wherein R5 is hydrogen atom or an alkyl group; R6 is hydrogen atom, an alkyl group or a thioalkyl group; X2 is a benzene ring or naphthalene ring each of which may be substituted with an alkyl group; Y is a benzene ring, hydroxyl benzene ring or a biphenyl ring each of which may be substituted with an alkyl group; j and k are each independently number of 1 to 15 as average value; and m is an integer of 1 to 5.

(3) The epoxy resin composition of the above-mentioned (1), wherein the phenolic curing agent (B) is a phenolic curing agent represented by formula (1′) or (2′): 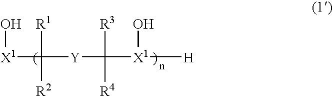
wherein R1 to R4 are each independently a hydrogen atom or an alkyl group, X1 is a benzene ring, hydroxyl benzene ring, naphthalene ring or hydroxyl naphthalene ring, each optionally substituted by an alkyl group, Y is a benzene ring, a hydroxybenzene ring or a biphenyl ring, each optionally substituted by an alkyl group, and n is a number of 1 to 15 as average value, 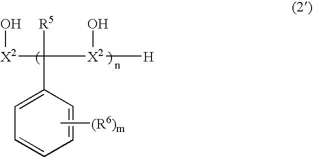
wherein R5 is a hydrogen atom or an alkyl group, R6 is a hydrogen atom, an alkyl group or a thioalkyl group, X2 is a benzene ring or a naphthalene ring, each optionally substituted by an alkyl group, n is a number of 1 to 15 as average value, and m is an integer of 1 to 5.

(4) The epoxy resin composition of the above-mentioned (1), wherein the phenolic curing agent (B) is a phenolic curing agent represented by any of formulas (3) to (5): 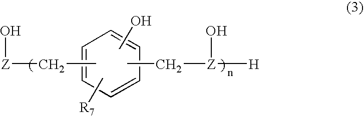
wherein R7 is a hydrogen atom or a methyl group, Z is a naphthalene ring, and n is a number of 1 to 15 as average value; 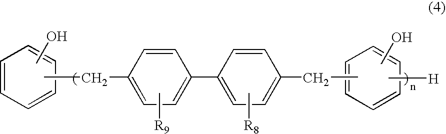
wherein R8 and R9 are each independently a hydrogen atom or a methyl group, and n is a number of 1 to 15 as average value; 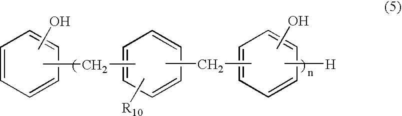
wherein R10 is a hydrogen atom or a methyl group, and n is a number of 1 to 15 as average value.

(5) The epoxy resin composition of the above-mentioned (1), wherein the phenolic curing agent (B) is a phenolic curing agent represented by formula (6): 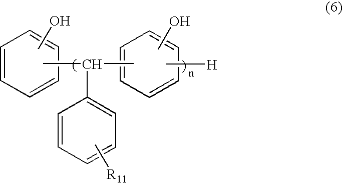
wherein R11 is a hydrogen atom, a methyl group, or a thiomethyl group, and n is a number of 1 to 15 as average value.

(6) The epoxy resin composition according to the above (1), wherein the phenol type curing agent of the component (B) is a phenol type curing agent represented by formula (7): 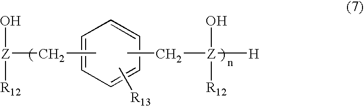
wherein R12 is hydrogen atom, a methyl group or hydroxyl group; R13 is hydrogen atom, a methyl group, or hydroxyl group; Z is a naphthalene ring; and n is a number of 1 to 15 as average value.

(7) The epoxy resin composition of any one of the above-mentioned (1) to (6), wherein the epoxy resin (A) comprises:

(A1) a first epoxy resin that is an aromatic epoxy resin having two or more epoxy groups in one molecule, which is liquid at a temperature of 20° C.; and

(A2) a second epoxy resin that is an aromatic epoxy resin having three or more epoxy groups in one molecule, which is solid at a temperature of 20° C.

(8) The epoxy resin composition of the above-mentioned (7), wherein the second epoxy resin (A2) has an epoxy equivalent weight of not more than 230.

(9) The epoxy resin composition of the above-mentioned (7), wherein the second epoxy resin (A2) has an epoxy equivalent weight within the range of 150 to 230.

(10) The epoxy resin composition of any one of the above-mentioned (7) to (9), comprising 0.3 to 2 parts by weight of the second epoxy resin (A2) per 1 part by weight of the first epoxy resin (A1).

(11) The epoxy resin composition of any one of the above-mentioned (1) to (10), wherein the polyvinyl acetal resin (C) has a glass transition temperature of not less than 80° C.

(12) The epoxy resin composition of any one of the above-mentioned (1) to (11), wherein the epoxy resin composition comprises 10 to 50 parts by mass of the epoxy resin (A) and 2 to 20 parts by mass of the polyvinyl acetal resin (C) per the total 100 parts by mass of the nonvolatile component, and

a number (E) of the epoxy group and a number (P) of the phenolic hydroxyl group of the phenolic curing agent (B), P/E, present in the epoxy resin composition is 0.5 to 1.5.

(13) The epoxy resin composition of any one of the above-mentioned (1) to (12), which further comprises:

(D) a phenoxy resin.

(14) The epoxy resin composition of the above-mentioned (13), which comprises 1 to 20 parts by mass of the phenoxy resin per 100 parts by mass of a nonvolatile component in the epoxy resin composition.

(15) The epoxy resin composition of any one of the above-mentioned (1) to (14), further comprising:

(D′) an inorganic filler.

(16) The epoxy resin composition of the above-mentioned (15), which comprises 10 to 75 parts by mass of the inorganic filler per 100 parts by mass of the nonvolatile component in the epoxy resin composition.

(17) An adhesive film, comprising a layer which comprises an epoxy resin composition of any one of the above-mentioned (1) to (16), which is formed on a support film.

(18) A prepreg, comprising a sheet-like reinforcement substrate made of a fiber, which is impregnated with a epoxy resin composition of any one of the above-mentioned (1) to (16).

(19) A multi-layer printed wiring board comprising an insulating layer obtained by curing an epoxy resin composition of any one of the above-mentioned (1) to (16).

(20) A production method of a multi-layer printed wiring board, which comprises:

(1) forming an insulating layer on an inner layer circuit substrate by heat curing an epoxy resin composition of any one of the above-mentioned (1) to (16);

(2) roughening the surface of the insulating layer; and

(3) forming a conductive layer on the roughened surface by copper plating step.

(21) A production method of a multi-layer printed wiring board, which comprises:

(1) forming an insulating layer on an inner layer circuit substrate by laminating an adhesive film of the above-mentioned (17) on the inner layer circuit substrate and heat curing the epoxy resin composition with or without delamination of the above-mentioned support film, after which delaminating the support film when the support film is present;

(2) roughening the surface of the insulating layer; and

(3) forming a conductive layer on the roughened surface by copper plating.

(22) A production method of a multi-layer printed wiring board, which comprises:

(1) forming an insulating layer on an inner layer circuit substrate by laminating a prepreg of the above-mentioned (18) on the inner layer circuit substrate and heat curing the epoxy resin composition;

(2) roughening the surface of the insulating layer; and

(3) forming a conductive layer on the roughened surface by copper plating.

(23) The production method of any one of the above-mentioned (20) to (22), wherein an oxidant is used in the roughening treatment.

(24) The production method of any one of the above-mentioned (20) to (22), wherein an alkaline permanganate solution is used in the roughening treatment.

(25) The production method of any one of the above-mentioned (20) to (24), wherein the roughened surface obtained by roughening the surface of the insulating layer has a roughness in an Ra value of not more than 0.5 μm and the conductive layer formed by plating has a peel strength of not less than 0.6 kgf/cm.

According to the present invention, although the roughness of the roughened surface after the roughening treatment is comparatively small, an insulating layer superior in the cohesive strength with the conductive layer formed by plating can be conveniently introduced into a multi-layer printed wiring board. That is, when a multi-layer printed wiring board is produced by introducing an insulating layer made of a cured product of the resin composition of the present invention, a roughened surface obtained by a roughening treatment of an insulating layer with an oxidant can maintain a plated conductor (conductive layer) with a highly cohesive force, even though the roughness is comparatively small. As a result, the flash etching for removing the unnecessary part of the plated conductive layer to complete the wiring formation can be performed in a short time. Therefore, a highly reliable, highly dense wiring that closely adheres to an insulating layer with a high cohesive strength (peel strength) and is free of damage or breakage can be formed with good reproducibility.

### DETAILED DESCRIPTION OF THE PREFERRED EMBODIMENTS

The present invention is explained in more detail in the following. Epoxy resin (A):

In the present invention, the “epoxy resin (A) having two or more epoxy groups in one molecule” is not particularly limited and, for example, cresol novolac epoxy resin, phenol novolac epoxy resin, tert-butyl-catechol epoxy resin, biphenyl epoxy resin, bisphenol A epoxy resin, bisphenol F epoxy resin, naphthalene epoxy resin, linear aliphatic epoxy resin, alicyclic epoxy resin, glycidyl amine epoxy resin, heterocyclic epoxy resin, spiro ring-containing epoxy resin, halogenated epoxy resin, and the like can be mentioned.

While one kind of epoxy resin (A) may be used alone or two or more kinds thereof may be used in combination, an embodiment using “a first epoxy resin (A1) that is an aromatic epoxy resin having two or more epoxy groups in one molecule, which is liquid at a temperature of 20° C.” and “a second epoxy resin (A2) that is an aromatic epoxy resin having three or more epoxy groups in one molecule, which is solid at a temperature of 20° C.” in combination is preferable. As the second epoxy resin, one having an epoxy equivalent weight of not more than 230 is more preferable, and one having an epoxy equivalent weight within the range of 150 to 230 is particularly preferable. The aromatic epoxy resin in the present invention is an epoxy resin having an aromatic ring skeleton in its molecule. The epoxy equivalent weight (g/eq) is molecular weight per one epoxy group.

Using, as the epoxy resin (A), the first epoxy resin (A1) and the second epoxy resin (A2), when the resin composition is used in the form of an adhesive film, an easy-to-handle adhesive film having sufficient flexibility can be formed and, simultaneously, the breaking strength of a cured product of the resin composition is increased and the durability of a multi-layer printed wiring board is increased.

In the present invention, as “the first epoxy resin (A1) that is an aromatic epoxy resin having two or more epoxy groups in one molecule, which is liquid at a temperature of 20° C.”, bisphenol A epoxy resin, bisphenol F epoxy resin, phenol novolac epoxy resin, tert-butyl-catechol epoxy resin, naphthalene epoxy resin, glycidyl amine epoxy resin, and the like can be mentioned. In the present invention, one kind of the first epoxy resin (A1) may be used or two or more kinds thereof may be used in combination. The first epoxy resin (A1) may be a liquid at a temperature of less than 20° C.

As such epoxy resin, specifically, HP4032 (manufactured by Dainippon Ink and Chemicals Incorporated), HP4032D (manufactured by Dainippon Ink and Chemicals Incorporated), jER807 (Epikote807) (manufactured by Japan Epoxy Resins Co., Ltd.), jER828EL (Epikote828) (manufactured by Japan Epoxy Resins Co., Ltd.), jER152 (Epikotel52) (manufactured by Japan Epoxy Resins Co., Ltd.), and the like can be mentioned.

On the other hand, as “the second epoxy resin (A2) that is an aromatic epoxy resin having three or more epoxy groups in one molecule, which is solid at a temperature of 20° C.”, for example, naphthalene epoxy resin, epoxy product of a condensation product of phenol and aromatic aldehyde having a phenolic hydroxyl group (trisphenol epoxy resin) and the like can be mentioned. For enhancement of the properties of the resin composition, such as glass transition temperature and the like, the second epoxy resin (A2) preferably has an epoxy equivalent weight of not more than 230, more preferably within the range of 150 to 230. Accordingly, in the present invention, the second epoxy resin (A2) is preferably “an aromatic epoxy resin that is an aromatic epoxy resin having three or more epoxy groups in one molecule and an epoxy equivalent weight of not more than 230, which is solid at a temperature of 20° C.” and more preferably “an aromatic epoxy resin that is an aromatic epoxy resin having three or more epoxy groups in one molecule and an epoxy equivalent weight of 150 to 230, which is solid at a temperature of 20° C.”. One kind of the second epoxy resin (A2) may be used or two or more kinds thereof may be used in combination. The second epoxy resin (A2) may be a solid at a temperature exceeding 20° C.

As such epoxy resin, specifically, HP4700 (EXA4700) (tetrafunctional naphthalene epoxy resin, epoxy equivalent weight 163, solid), N-690 (cresol novolac epoxy resin, epoxy equivalent weight 208, solid) and N-695 (cresol novolac epoxy resin, epoxy equivalent weight 208, solid) manufactured by Dainippon Ink and Chemicals Incorporated, EPPN-502H (trisphenol epoxy resin, epoxy equivalent weight 168, solid), NC7000L (naphthol novolac epoxy resin, epoxy equivalent weight 228, solid) and NC3000H (biphenyl epoxy resin, epoxy equivalent weight 290, solid) of Nippon Kayaku Co., Ltd., ESN185 (naphthol novolac epoxy resin, epoxy equivalent weight 275, solid) and ESN475 (naphthol novolac epoxy resin, epoxy equivalent weight 350, solid) manufactured by Tohto Kasei Co., Ltd., and the like can be mentioned.

When the first epoxy resin (A1) and the second epoxy resin (A2) are used in combination as the epoxy resin (A), 0.3 to 2 parts by weight, more preferably 0.5 to 1 part by weight of the second epoxy resin (A2), is preferably used per 1 part by weight of the first epoxy resin (A1). When the proportion of the first epoxy resin (A1) is too high, the adhesiveness of the resin composition becomes high, degassing property becomes low during vacuum lamination when it is used in the form of an adhesive film, and voids tend to be easily developed. In addition, the releaseability of the protection film and support film during vacuum lamination as well as heat resistance after curing tend to decrease. Furthermore, cured products of the resin composition tend to show insufficient breaking strength. When the proportion of the second epoxy resin (A2) is too high, there is a propensity that sufficient flexibility is not afforded, handling property is degraded, sufficient flowability cannot be obtained easily during lamination and the like, when it is used in the form of an adhesive film.

In the epoxy resin composition of the present invention, epoxy resin (A) is contained in the proportion of 10 to 50 parts by mass, more preferably 20 to 40 parts by mass, still more preferably 20 to 35 parts by mass, relative to the total 100 parts by mass of the nonvolatile component of the epoxy resin composition. The curing property of the resin composition is not degraded easily within the aforementioned range. The epoxy resin composition of the present invention may contain epoxy resin other than component (A) as long as the effect of the invention can be afforded. The amount of the epoxy resin in 100 parts by mass of the nonvolatile component of the epoxy resin composition is generally not more than 50 parts by mass.

Phenolic Curing Agent (B):

The phenolic curing agent to be used in the present invention has an average content ratio of hydroxyl group in one molecule (P) of 0<P<1. Such phenolic curing agents can be used without any particularly limitation. The average content ratio of hydroxyl group in one molecule (P) is an average value of H/B wherein H is the total number of hydroxyl groups and B is the total number of benzene rings. The scope of the average content ratio of hydroxyl group in one molecule (P) is 1/4<P<9/10 is preferable, and furthermore 1/3≦P≦4/5 is more preferable. As used herein, the “phenolic curing agent” refers to a compound having 2 or more phenolic hydroxyl groups in one molecule, which acts as a curing agent of epoxy resin (A).

As the “phenolic curing agent having an average content ratio of hydroxyl group in one molecule (P) of 0<P<1”, a phenolic curing agent represented by the following formula (1) or (2) is preferable. 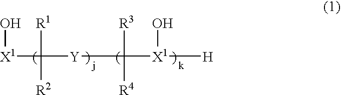
wherein R¹ to R⁴ are each independently hydrogen atom or an alkyl group; X¹ is a benzene ring, hydroxyl benzene ring, naphthalene ring or hydroxyl naphthalene ring each of which may be substituted with an alkyl group; Y is a benzene ring, hydroxyl benzene ring or a biphenyl ring each of which may be substituted with an alkyl group; and j and k are each independently number of 1 to 15 as average value. 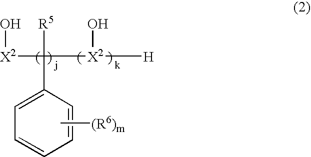
wherein R5 is a hydrogen atom or an alkyl group, R6 is a hydrogen atom, an alkyl group or a thioalkyl group, X2 is a benzene ring or a naphthalene ring, each optionally substituted by an alkyl group, and j and k are each independently number of 1 to 15 as average value, and m is an integer of 1 to 5.

In the formula (2), when m is 2 to 5, plural R6 do not need to be the same, but may be each independently a group selected from hydrogen atom, alkyl group and thioalkyl group.

For the phenolic curing agent represented by formula (1) and (2), arrangement of each unit in parentheses may be random as long as satisfying the condition that j and k is a number of 1 to 15 as average value. Each unit is not necessary to link between the same units successionally, and each unit can link together alternately or randomly.

As the alkyl group, an alkyl group having 1 to 3 carbon atoms is preferable, and a methyl group is particularly preferable. As the thioalkyl group, a thioalkyl group having 1 to 3 carbon atoms is preferable, and a thiomethyl group is preferable.

As the “phenolic curing agent having an average content ratio of hydroxyl group in one molecule (P) of 0<P<1”, a phenolic curing agent represented by the following formula (1′) or (2′) is more preferable. 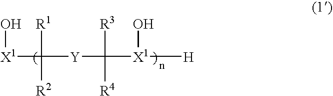
wherein R1 to R4 are each independently a hydrogen atom or an alkyl group, X1 is a benzene ring, hydroxyl benzene ring, naphthalene ring or hydroxyl naphthalene ring, each optionally substituted by an alkyl group, Y is a benzene ring, a hydroxybenzene ring or a biphenyl ring, each optionally substituted by an alkyl group, and n is a number of 1 to 15 as average value. 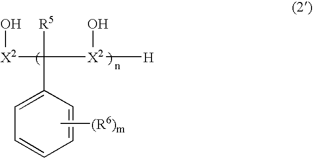
wherein R5 is a hydrogen atom or an alkyl group, R6 is a hydrogen atom, an alkyl group or a thioalkyl group, X2 is a benzene ring or a naphthalene ring, each optionally substituted by an alkyl group, n is a number of 1 to 15 as average value, and m is an integer of 1 to 5.

In the formula (2′), when m is 2-5, plural R6 do not need to be the same, but may be each independently a group selected from hydrogen atom, alkyl group and thioalkyl group.

As the alkyl group in the formula (1′) and (2′), an alkyl group having 1-3 carbon atoms is preferable, and a methyl group is particularly preferable. As the thioalkyl group in the formula (2′), a thioalkyl group having 1-3 carbon atoms is preferable, and a thiomethyl group is particularly preferable.

As the “phenolic curing agent having an average content ratio of hydroxyl group in one molecule (P) of 0<P<1”, particularly, a phenolic curing agent represented by the following formula (3) to (7) is preferable. 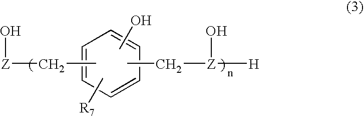
wherein R7 is a hydrogen atom or a methyl group, Z is a naphthalene ring, and n is a number of 1 to 15 as average value. 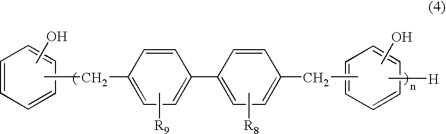
wherein R8 and R9 are each independently a hydrogen atom or a methyl group, and n is a number of 1 to 15 as average value. 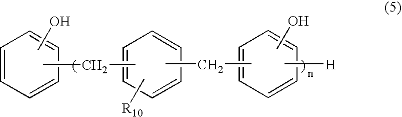
wherein R10 is a hydrogen atom or a methyl group, and n is a number of 1 to 15 as average value. 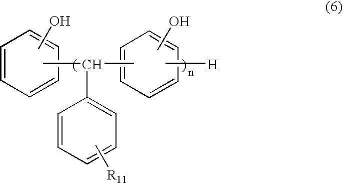
wherein R11 is a hydrogen atom, a methyl group, or a thiomethyl group, and n is a number of 1 to 15 as average value. 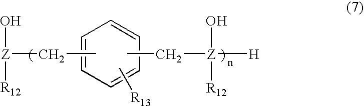
wherein R12 is a hydrogen atom, a methyl group or hydroxyl group, R13 is a hydrogen atom or a methyl group, Z is a naphthalene ring, n is a number of 1 to 15 as average value.

Specifically preferable examples of the phenolic curing agent are as follows. As the phenolic curing agent represented by the formula (3), NHN (Z: naphthalene ring, R7: methyl group, average hydroxyl group content ratio: 3/5˜2/3, see figure below) and CBN (Z: naphthalene ring, R7: methyl group, average hydroxyl group content ratio: 3/5˜2/3, see figure below) manufactured by Nippon Kayaku Co., Ltd. can be mentioned. As the phenolic curing agent represented by the formula (4), GPH103 (R8 and R9: hydrogen atom, average hydroxyl group content ratio: 1/3˜1/2) manufactured by Nippon Kayaku Co., Ltd. and MEH7851 (R8 and R9: hydrogen atom, average hydroxyl group content ratio: 1/3˜1/2) of Meiwa Plastic Industries, Ltd. can be mentioned. As the phenolic curing agent represented by the formula (5), MEH7800 (R10: hydrogen atom, average hydroxyl group content ratio: 1/3˜1/2) of Meiwa Plastic Industries, Ltd., XL225 (R10: hydrogen atom, average hydroxyl group content ratio: 1/3˜1/2) manufactured by Mitsui Chemicals, Inc. can be mentioned. As the phenolic curing agent represented by the formula (6), YLH1027 (R10: hydrogen atom, average hydroxyl group content ratio: 1/3˜1/2) of Japan Epoxy Resins Co., Ltd. and YLH1110L (R11: thiomethyl group, average hydroxyl group content: 1/3˜1/2) can be mentioned. In addition, as the phenolic curing agent represented by the formula (7), SN170, SN180, SN190 (R12 and R13: hydrogen atoms, average hydroxyl group content ratio: 1/3˜2/5, see figure below, softening points are 70° C., 80° C., 90° C. independently), SN475, SN485, SN495 (R12 and R13: hydrogen atoms, average hydroxyl group content ratio: 1/3˜2/5, see figure below, softening points are 75° C., 85° C., 95° C. independently), SN375 and SN395 (R12:hydroxyl group, R13: hydrogen atom, average hydroxyl group content ratio: 2/3˜4/5, see figure below, softening points are 75° C., 95° C. independently) manufactured by Tohto Kasei Co., Ltd can be mentioned. In the present invention, one kind of the phenolic curing agent (B) may be used or two or more kinds thereof may be used in combination.

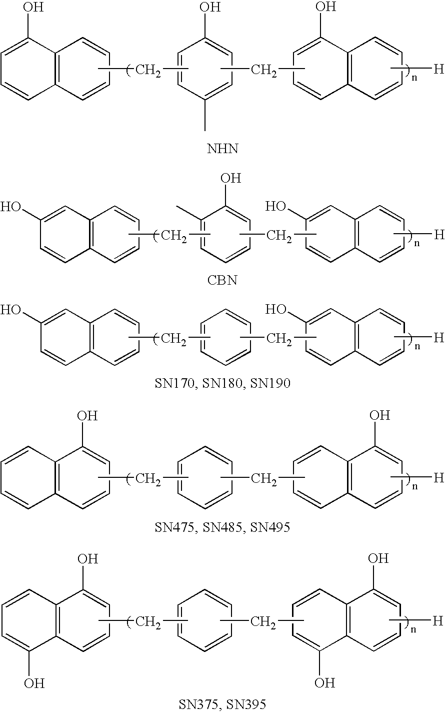

The resin composition of the present invention may contain a phenolic curing agent other than the above mentioned phenolic curing agent (B). In this case, to sufficiently show the effect of the invention, preferably not less than 50 mass %, more preferably not less than 70 mass %, particularly not less than 90 mass %, of the whole phenolic curing agents in the composition is preferably phenolic curing agent (B).

In the present invention, the amount of the phenolic curing agent in the resin composition is generally such an amount that achieves ratio P/E of preferably 0.5 to 1.5, more preferably 0.5 to 1.0, wherein E is the total number of the epoxy groups present in the resin composition and P is the total number of the phenolic hydroxyl groups in the phenolic curing agent. The amount of the phenolic curing agent means the total amount of phenolic curing agent (B) when the phenolic curing agent (B) alone is used and, when a phenolic curing agent other than the phenolic curing agent (B) and the phenolic curing agent (B) is used in combination, the total amount thereof. The total number of epoxy groups in the resin composition is the total of the values of all epoxy resins, which is obtained by dividing the solid content mass of each epoxy resin by the epoxy equivalent weight. The total number of the phenolic hydroxyl groups in the phenolic curing agent is the total of the values of all phenolic curing agents, which is obtained by dividing the solid content mass of each phenolic curing agent by the phenolic hydroxyl group equivalent. When the content of the phenolic curing agent is outside the preferable ranges, the heat resistance of the cured product obtained by curing the resin composition sometimes becomes insufficient.

Polyvinyl Acetal Resin (C):

While the polyvinyl acetal resin in the present invention is not particularly limited, a polyvinyl butyral resin is preferable. Specific examples of the polyvinyl acetal resin include Denka Butyral 4000-2, 5000-A, 6000-C and 6000-EP manufactured by Denki Kagaku Kogyo Kabushiki Kaisha, S-LEC BH series, BX series, KS series, BL series and BM series manufactured by Sekisui Chemical Co., Ltd., and the like.

A polyvinyl acetal having a glass transition temperature of not less than 80° C. is particularly preferable. The “glass transition temperature” here is determined according to the method described in JIS K 7197. When the glass transition temperature cannot be actually observed since it is higher than the decomposition temperature, the decomposition temperature can be considered the glass transition temperature in the present invention. The decomposition temperature is defined to be a temperature at which the mass decrease rate as measured according to the method described in JIS K 7120 is 5%.

The polyvinyl acetal resin (C) is preferably contained in a proportion of 2 to 20 parts by mass of the total 100 parts by mass of the nonvolatile component in the epoxy resin composition of the present invention. When it is less than 2 parts by mass, the peel strength of the conductive layer formed by plating tends to be insufficient, and when it exceeds 20 parts by mass, the roughness tends to be too high.

The epoxy resin composition of the present invention may contain an inorganic filler to decrease the coefficient of thermal expansion and the like. As the inorganic filler, for example, silica, alumina, barium sulfate, talc, clay, mica powder, aluminum hydroxide, magnesium hydroxide, calcium carbonate, magnesium carbonate, magnesium oxide, boron nitride, aluminum borate, barium titanate, strontium titanate, calcium titanate, magnesium titanate, bismuth titanate, titanium oxide, barium zirconate, calcium zirconate, and the like can be mentioned, with particular preference given to silica. The inorganic filler preferably has an average particle size of not more than 1 μm, more preferably not more than 0.8 μm, particularly preferably not more than 0.7 μm. When the average particle size exceeds 1 μm, the peel strength of the conductive layer formed by plating tends to decrease. When the average particle size of the inorganic filler becomes too small and when the epoxy resin composition is made into a resin varnish, the varnish shows an increased viscosity, which tends to degrade the handling property. Thus, the average particle size is preferably not less than 0.05 μm. Since inorganic filler increases moisture resistance, an inorganic filler surface-treated with a surface treating agent such as silane coupling agent and the like is preferable.

The average particle size of the above-mentioned inorganic filler can be measured by the laser diffraction and scattering method based on the Mie scattering theory. Specifically, the particle size distribution of the inorganic filler by volume standard is depicted using a laser diffraction type particle size distribution measurement device, and the median diameter thereof is taken as the average particle size. As a measurement sample, an inorganic filler is dispersed in water by ultrasonication and can be preferably used. As the laser diffraction type particle size distribution measurement device, LA-500 (Horiba Ltd.) and the like can be used.

When the inorganic filler is added, its content varies depending on the required properties. The content is preferably 10 to 75 parts by mass, more preferably 20 to 50 parts by mass, particularly preferably 20 to 40 parts by mass, per 100 parts by mass of the nonvolatile component in the epoxy resin composition of the present invention.

The epoxy resin composition of the present invention may contain solid rubber particles to enhance the mechanical strength of cured products, afford a stress relaxation effect and the like. The rubber particles in the present invention are not dissolved in an organic solvent during preparation of an epoxy resin composition, and are not compatible with the components in the resin composition such as epoxy resin and the like. Accordingly, the rubber particles in the present invention are present in a dispersion state in a varnish made from the epoxy resin composition. Such rubber particles are generally prepared by increasing the molecular weight of the rubber component to the extent that prevents dissolution in an organic solvent and resin, and processing the component into particles. As the rubber particles, for example, core-shell type rubber particles, crosslinked acrylonitrile-butadiene rubber particles, crosslinked styrene-butadiene rubber particles, acrylic rubber particles and the like can be mentioned. The core-shell type rubber particles have a core layer and a shell layer and, for example, a two-layer structure wherein the outer shell layer is a glass polymer, and the inner core layer is a rubber polymer, a three-layer structure wherein the outer shell layer is a glass polymer, the intermediate layer is a rubber polymer, and the core layer is a glass polymer, and the like can be mentioned. The glass layer consists of, for example, a methyl methacrylate polymer and the like, and the rubber polymer layer consists of, for example, a butylacrylate polymer (butyl rubber) and the like. Specific examples of the core-shell type rubber particles include Stafyloid AC3832, AC3816N, (Ganz Chemical Co., Ltd. trade name) and Metablen KW-4426 (Mitsubishi Rayon Co., Ltd. trade name). Specific examples of the acrylonitrile-butadiene rubber (NBR) particles include XER-9 1 (average particle size 0.5 μm, manufactured by JSR Corporation) and the like. Specific examples of the styrene-butadiene rubber (SBR) particles include XSK-500 (average particle size 0.5 μm, manufactured by JSR Corporation) and the like. Specific examples of the acrylic rubber particles include Metablen W300A (average particle size 0.1 μm), W450A (average particle size 0.5 μm) (manufactured by Mitsubishi Rayon Co., Ltd.).

The average particle size of the rubber particles to be added is preferably within the range of 0.005 to 1 μm, more preferably 0.2 to 0.6 μm. The average particle size of the rubber particles in the present invention can be measured using the dynamic light scattering method. For example, rubber particles are uniformly dispersed in a suitable organic solvent by ultrasonication and the like, the particle size distribution of the rubber particles by mass standard is depicted using FPRA-1000 (manufactured by Otsuka Electronics Co., Ltd.), and the median diameter thereof is taken as the average particle size.

When rubber particles are added, their content is preferably 1 to 10 mass %, more preferably 2 to 5 mass %, relative to the resin composition (nonvolatile component 100 mass %).

The epoxy resin composition of the present invention may contain a phenoxy resin to impart sufficient flexibility to an adhesive film, and the like. The content of the phenoxy resin per total 100 parts by mass of the nonvolatile component in the epoxy resin composition is preferably 1 to 20 parts by mass. Specific examples of the phenoxy resin include FX280 and FX293 manufactured by Tohto Kasei Co., Ltd., YX8100, YX6954(YL6954) and YL6974 manufactured by Japan Epoxy Resins Co., Ltd., and the like.

The epoxy resin composition of the present invention may contain a curing accelerator to adjust the curing time and the like. As the curing accelerator, for example, organic phosphine compound, imidazole compound, amine adduct compound, tertiary amine compound, and the like can be mentioned. Specific examples of the organic phosphine compound include TPP, TPP-K, TPP-S, and TPTP-S (Hokko Chemical Industry Co., Ltd. trade names), and the like. Specific examples of the imidazole compound include curezol2MZ, 2E4MZ, C11Z, C11Z-CN, C11Z-CNS, C11Z-A, 2MZ-OK, 2MA-OK, and 2PHZ (Shikoku Chemicals Corporation, trade names), and the like. Specific examples of the amine adduct compound include Novacure (Asahi Chemical Industry Co., Ltd., trade name), Fujicure (Fuji Kasei Kogyo Co., Ltd., trade name), and the like. Specific examples of the tertiary amine compound include DBU (1,8-diazabicyelo[5,4,0]undec-7-ene) and the like. In the epoxy resin composition of the present invention, the content of the curing accelerator is generally within the range of 0.1 to 5 parts by mass relative to the total 100 parts by mass of the epoxy resin and the solid content of the phenolic curing agent contained in the epoxy resin composition.

The epoxy resin composition of the present invention may contain a flame retardant to impart flame retardance. As the flame retardant, for example, organic phosphorus flame retardant, organic nitrogen-containing phosphorus compound, nitrogen compound, silicone flame retardant, metal hydroxide, and the like can be mentioned.

As the organic phosphorus flame retardant, phosphine compounds such as HCA, HCA-HQ, and HCA-NQ manufactured by Sanko Co., Ltd., and the like, phosphate ester compounds such as Reofos 30, 50, 65, 90, 110, TPP, RPD, BAPP, CPD, TCP, TXP, TBP, TOP, KP140, and TIBP manufactured by Ajinomoto-Fine-Techno Co., Inc., PPQ manufactured by Hokko Chemical Industry Co., Ltd., OP930 manufactured by Clariant K.K., PX200 manufactured by Daihachi Chemical Industry Co., Ltd., and the like, phosphorus containing epoxy resins such as FX289 and FX310 manufactured by Tohto Kasei Co., Ltd., and the like, phosphorus containing phenoxy resins such as ERF001 manufactured by Tohto Kasei Co., Ltd., and the like, and the like can be mentioned.

As the organic nitrogen-containing phosphorus compound, phosphate ester amide compounds such as SP670 and SP703 manufactured by Shikoku Chemicals Corporation, and the like, phosphagen compounds such as SPB100 and SPE100 manufactured by OTSUKA Chemical Co., Ltd., and the like, and the like can be mentioned.

As the metal hydroxide, magnesium hydroxide such as UD65, UD650, and UD653 manufactured by Ube Material Industries, Ltd., and the like, aluminum hydroxide such as B-30, B-325, B-315, B-308, B-303, and UFH-20 manufactured by Tomoe Engineering Co., Ltd., and the like, and the like can be mentioned.

When a flame retardant is added, its content relative to the resin composition (nonvolatile component 100 mass %) is preferably not more than 20 mass %, more preferably not more than 15 mass %.

The epoxy resin composition of the present invention may contain a resin additive other than those mentioned above as long as the effect of the invention can be afforded. As the resin additive, for example, organic fillers such as silicone powder, nylon powder, fluorine powder, and the like, thickeners such as orben, benton and the like, silicone, fluorine and polymer antifoaming agents or leveling agents, tackifiers such as imidazole, thiazole, triazole, and silane coupling agents and the like, coloring agents such as phthalocyanine-blue, phthalocyanine-green, iodine-green, disazo yellow, carbon black and the like, and the like can be mentioned.

The resin composition of the present invention can be processed into an adhesive film or prepreg for an interlayer insulating layer of a multi-layer printed wiring board, by applying the composition on a support film, thus forming a resin composition layer to give an adhesive film for a multi-layer printed wiring board, or impregnating a sheet-like reinforcement substrate made of a fiber with the resin composition to afford a prepreg. While the resin composition of the present invention can be applied to a circuit substrate to form an insulating layer, industrially, it is generally used in the form of an adhesive film or a prepreg for forming an insulating layer.

The adhesive film of the present invention can be produced by a method known to those of ordinary skill in the art; for example, by dissolving a resin composition in an organic solvent to give a resin varnish, coating a support film as a support with the resin varnish, and evaporating the organic solvent by heating or blowing hot air and the like to form a resin composition layer.

As the organic solvent, for example, ketones such as acetone, methylethylketone, cyclohexanone, and the like, acetate esters such as ethyl acetate, butyl acetate, cellosolve acetate, propyleneglycol monomethylether acetate, carbitol acetate, and the like, carbitols such as cellosolve, butyl carbitol, and the like, aromatic hydrocarbons such as toluene, xylene, and the like, amide solvents such as dimethylformamide, dimethylacetamide, N-methylpyrrolidone, and the like, and the like can be mentioned. One kind of an organic solvent may be used or two or more kinds thereof may be used in combination.

While the drying conditions are not particularly limited, the film is dried so that the content of the organic solvent in the resin composition layer will be generally not more than 10 mass %, preferably not more than 5 mass %. The drying conditions can be appropriately set to be preferable drying conditions by a simple experiment. While subject to variation depending on the amount of the organic solvent in a varnish, for example, a varnish containing 30 to 60 mass % of an organic solvent can be dried at 50 to 150° C. for about 3 to 10 minutes.

The thickness of the resin composition layer to be formed in an adhesive film is generally not less than the thickness of the conductive layer. Since the thickness of a conductive layer of a circuit substrate is generally within the range of 5 to 70 μm, the thickness of the resin composition layer is preferably 10 to 100 μm. The resin composition layer may be protected by the below-mentioned protection film. By protecting with a protection film, attachment of dust and the like to the surface of a resin composition layer and scar thereon can be prevented.

As the support film and protection film in the present invention, polyolefin such as polyethylene, polypropylene, polyvinyl chloride, and the like, polyethylene terephthalate (hereinafter sometimes to be abbreviated as “PET”), polyester such as polyethylene naphthalate and the like, polycarbonate, polyimide, release paper, metal foil such as copper foil, aluminum foil, and the like, and the like can be mentioned. The support film and protection film may be subjected to a release treatment, in addition to a matte treatment and a corona treatment.

While the thickness of the support film is not particularly limited, it is generally 10 to 150 μm, preferably within the range of 25 to 50 μm. While the thickness of the protection film is not particularly limited, it is generally 1 to 40 μm, preferably within the range of 10 to 30 μm. As mentioned later, the support film to be used as a support in the production step of an adhesive film can also be used as a protection film to cover the surface of a resin composition layer.

The support film in the present invention is delaminated after lamination on a circuit substrate or after formation of an insulating layer by heat curing. When the support film is delaminated after heat curing the adhesive film, attachment of dust and the like during the curing step can be prevented, and smoothness of the surface of the insulating layer after curing can be improved. For delamination after curing, a release treatment is generally applied in advance to the support film. The resin composition layer to be formed on the support film preferably has a smaller area than the area of the support film. The adhesive film can be wound in a roll and preserved or stored.

A method of producing the multi-layer printed wiring board of the present invention using the adhesive film of the present invention is explained in the following. When a resin composition layer is protected with a protection film(s), they are delaminated, and the resin composition layer is laminated on one surface or both surfaces of the circuit substrate to be in direct contact therewith. The adhesive film of the present invention is preferably laminated on a circuit substrate under reduced pressure by a vacuum lamination method. The lamination method may be a batch method or a continuation method using rolls. Where necessary, the adhesive film and the circuit substrate may be heated (preheated) before lamination.

The lamination conditions preferably include a press temperature (lamination temperature) of preferably 70 to 140° C., a press pressure of preferably 1 to 11 kgf/cm² (9.8×10⁴ to 107.9×10⁴ N/m²), and an air pressure of 20 mmHg (26.7 hPa) or below under reduced pressure.

Vacuum lamination can be performed using a commercially available vacuum laminator. As the commercially available vacuum laminator, for example, the vacuum applicator manufactured by Nichigo-Morton Co., Ltd., the vacuum pressure laminator manufactured by Meiki Co., Ltd., the roll dry coater manufactured by Hitachi Industries Co., Ltd., the vacuum laminator manufactured by Hitachi AIC Inc., and the like can be mentioned.

The circuit substrate in the present invention mainly refers to a substrate having a conductive layer (circuit) pattern formed on one surface or both surfaces such as glass epoxy, metal substrate, polyester substrate, polyimide substrate, BT resin substrate, heat curing type polyphenylene ether substrate, and the like. In addition, a multi-layer printed wiring board having a conductive layer and an insulating layer alternately formed in layers, and a conductive layer (circuit) pattern formed on one surface or both surfaces is encompassed in the circuit substrate in the present invention. The surface of the conductive circuit layer is preferably applied to a roughening treatment in advance by a blackening treatment and the like, from the aspect of cohesion of the insulating layer to the circuit substrate.

After lamination of the adhesive film on the circuit substrate, the support film is delaminated as necessary, and the laminate is heat cured to form an insulating layer on the circuit substrate. The heat curing conditions are selected from the range of 150° C. to 220° C. for 20 minutes to 180 minutes, more preferably 160° C. to 200° C. for 30 to 120 minutes.

After formation of the insulating layer, the support film is delaminated at this point when it was not delaminated before curing. Then the insulating layer formed on the circuit substrate is perforated to form via holes and through-holes. For perforation, for example, known methods such as drill, laser, plasma and the like can be employed, which may be combined as necessary. Perforation by laser such as carbon dioxide gas laser, YAG laser and the like is the most common method.

Then, the surface of the insulating layer is subjected to a roughening treatment. Generally, the roughening treatment in the present invention is preferably performed by a wet roughening method using an oxidant. As the oxidant, permanganate salt (potassium permanganate, sodium permanganate etc.), dichromate salt, ozone, hydrogen peroxide/sulfuric acid, nitric acid, and the like can be mentioned. Preferably, an alkaline permanganate solution (e.g., aqueous sodium hydroxide solutions of potassium permanganate, sodium permanganate) is used for roughening, which is an oxidant widely used for roughening of an insulating layer during production of a multi-layer printed wiring board by a build-up method.

The roughness of the roughened surface obtained by roughening the surface of the insulating layer is preferably not more than 0.5 μm, more preferably not more than 0.35 μm, in terms of its Ra value, for forming an ultrafine wiring.

Then, a conductive layer is formed on the surface of a resin composition layer, on which convex and concave anchors have been formed by the roughening treatment, by a combined method of electroless plating and electroplating. It is also possible to form a conductive layer by electroless plating alone by forming a plating resist pattern reverse to the conductive layer. The peel strength of the conductive layer can be further improved and stabilized by an anneal treatment at 150 to 200° C. for 20 to 90 minutes after formation of the conductive layer. According to the present invention, a preferable peel strength of the conductive layer for a multi-layer printed wiring board can be obtained. The peel strength preferable for a multi-layer printed wiring board is generally not less than 0.6 kgf/cm, more preferably not less than 0.7 kgf/cm, still more preferably not less than 0.8 kgf/cm.

As a method for forming a circuit pattern by processing the conductive layer, for example, a subtractive method, a semi-additive method and the like known to those of ordinary skill in the art can be used.

The prepreg of the present invention can be produced by impregnating a sheet-like reinforcement substrate made of a fiber with the resin composition of the present invention by a hotmelt method or a solvent method and semi-cured by heating. In other words, the prepreg comprises a sheet-like reinforcement substrate made of a fiber impregnated with the resin composition of the present invention.

As the sheet-like reinforcement substrate made of a fiber, for example, those conventionally used as a prepreg fiber such as glass cloth, aramid fiber, and the like can be employed.

The hotmelt method comprises first coating a resin on a paper showing good releaseability from the resin, without dissolving the resin in an organic solvent, and laminating same on a sheet-like reinforcement substrate, or directly applying a resin with a die coater and the like to produce a prepreg. The solvent method comprises, like adhesive films, immersing a sheet-like reinforcement substrate in a resin varnish obtained by dissolving the resin in an organic solvent, impregnating the sheet-like reinforcement substrate with the resin varnish, and drying the substrate.

A method of producing the multi-layer printed wiring board of the present invention using the prepreg of the present invention is explained in the following. One sheet or several sheets, where necessary, of the prepreg of the present invention is/are superimposed on a circuit substrate, a metal plate is sandwiched via a release film, and the laminate is pressed under pressurization and heating conditions. The pressure is preferably applied for molding at 5 to 40 kgf/cm² (49×10⁴ to 392×10⁴ N/m²) preferably a temperature of 120 to 200° C. for 20 to 100 minutes. It is also possible to produce a pressed laminate by laminating the prepreg on a circuit substrate by vacuum lamination as in the case of adhesive film, and then heat curing the lamination. Thereafter, in the same manner as in the aforementioned method, the prepreg surface cured with an oxidant is roughened and the conductive layer is formed by plating, whereby a multi-layer printed wiring board can be produced.

Other features of the invention will become apparent in the course of the following descriptions of exemplary embodiments which are given for illustration of the invention and are not intended to be limiting thereof.

### EXAMPLES

In the following examples, “parts” means “parts by mass”.

### Example 1

A polyvinyl butyral resin (glass transition temperature 105° C., “KS-1” manufactured by Sekisui Chemical Co., Ltd.) was dissolved in a 1:1 mixed solvent of ethanol and toluene at 60° C. to a solid content of 15% to give a polyvinyl butyral resin solution. Then, liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in methylethylketone (15 parts) (hereinafter to be abbreviated as “MEK”) and cyclohexanone (15 parts) by stirring with heating. Thereto were added a phenolic curing agent (an MEK solution (110 parts, solid content 50%) of a novolac resin having a naphthalene structure (“SN-485” manufactured by Tohto Kasei Co., Ltd., phenolic hydroxyl group equivalent amount 215, average hydroxyl group content ratio 1/3˜2/5)), a curing catalyst (“2E4MZ” manufactured by Shikoku Chemicals Corporation, 0.1 part), spherical silica (average particle size 0.5 μm, “SOC2” manufactured by Admatechs Corporation Limited, 70 parts), and the aforementioned polyvinyl butyral resin solution (35 parts), and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.78). The resin varnish was applied to polyethylene terephthalate (thickness 38 μm, hereinafter to be abbreviated as “PET”) with a die coater such that the resin thickness after drying was 40 μm, and dried at 80 to 120° C. (average 100° C.) for 6 minutes (amount of residual solvent, about 1 mass %). The resin composition was wound in a roll while adhering a 15 μm-thick polypropylene film to the surface of the resin composition. The adhesive film roll was slit in a width of 507 mm, from which a 507×336 mm sheet-like adhesive film was obtained.

### Example 2

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenolic curing agent (an MEK solution (75 parts, solid content 50%) of a novolac resin having a naphthalene structure (“CBN” manufactured by Nippon Kayaku Co., Ltd., phenolic hydroxyl group equivalent amount 139, average hydroxyl group content ratio 3/5˜2/3)), a curing catalyst (“2E4MZ” manufactured by Shikoku Chemicals Corporation, 0.1 part), spherical silica (average particle size 0.5 μm, “SOC2” manufactured by Admatechs Corporation Limited, 60 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.82). In the same manner as in Example 1, a sheet-like adhesive film was obtained.

### Example 3

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenolic curing agent (an MEK solution (75 parts, solid content 60%) of a novolac resin having a thiomethyl group (“YLH-1110 L” manufactured by Japan Epoxy Resins Co., Ltd., phenolic hydroxyl group equivalent amount 168, average hydroxyl group content ratio 1/2˜1/3)), a curing catalyst (“2E4MZ” manufactured by Shikoku Chemicals Corporation, 0.1 part), spherical silica (average particle size 0.5 μm, “SOC2” manufactured by Admatechs Corporation Limited, 60 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.82). In the same manner as in Example 1, a sheet-like adhesive film was obtained.

### Example 4

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenolic curing agent (an MEK solution (65 parts, solid content 60%) of a novolac resin (“YLH1027” manufactured by Japan Epoxy Resins Co., Ltd., phenolic hydroxyl group equivalent amount 120, average hydroxyl group content ratio 1/2˜1/3)), a curing catalyst (“2E4MZ” manufactured by Shikoku Chemicals Corporation, 0.1 part), spherical silica (average particle size 0.5 μm, “SOC2” manufactured by Admatechs Corporation Limited, 75 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.99). In the same manner as in Example 1, a sheet-like adhesive film was obtained.

### Example 5

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenolic curing agent (an MEK solution (120 parts, solid content 50%) of a novolac resin having a biphenyl structure (“GPH-103” manufactured by Nippon Kayaku Co., Ltd., phenolic hydroxyl group equivalent amount 231, average hydroxyl group content ratio 1/2˜1/3)), a curing catalyst (“2E4MZ” manufactured by Shikoku Chemicals Corporation, 0.1 part), spherical silica (average particle size 0.5 μm, “SOC2” manufactured by Admatechs Corporation Limited, 75 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.79). In the same manner as in Example 1, a sheet-like adhesive film was obtained.

### Example 6

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenolic curing agent (an MEK solution (75 parts, solid content 50%) of a novolac resin having a naphthalene structure (“NHN” manufactured by Nippon Kayaku Co., Ltd., phenolic hydroxyl group equivalent amount 143, average hydroxyl group content ratio 3/5˜2/3)), a curing catalyst (“2E4MZ” manufactured by Shikoku Chemicals Corporation, 0.1 part), spherical silica (average particle size 0.5 μm, “SOC2” manufactured by Admatechs Corporation Limited, 60 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.8). In the same manner as in Example 1, a sheet-like adhesive film was obtained.

### Example 7

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenoxy resin varnish (mixed solution of cyclohexanone containing a 30 mass % of a nonvolatile component and MEK, “YL6954BH30” manufactured by Japan Epoxy Resins Co., Ltd., glass transition temperature 130° C., 15 parts), a phenolic curing agent (an MEK solution (110 parts, solid content 50%) of a novolac resin having a naphthalene structure (“SN-485” manufactured by Tohto Kasei Co., Ltd., phenolic hydroxyl group equivalent amount 215, average hydroxyl group content ratio 1/3˜2/5)), a curing catalyst (“2E4MZ” manufactured by Shikoku Chemicals Corporation, 0.1 part), spherical silica (average particle size 0.5 μm, “SOC2” manufactured by Admatechs Corporation Limited, 75 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.78). In the same manner as in Example 1, a sheet-like adhesive film was obtained.

### Example 8

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenoxy resin varnish (mixed solution of cyclohexanone containing a 30 mass % of a nonvolatile component and MEK, “YL6954BH30” manufactured by Japan Epoxy Resins Co., Ltd., glass transition temperature 130° C., 15 parts), a phenolic curing agent (an MEK solution (75 parts, solid content 50%) of a novolac resin having a naphthalene structure (“CBN” manufactured by Nippon Kayaku Co., Ltd., phenolic hydroxyl group equivalent amount 139, average hydroxyl group content ratio 3/5˜2/3)), a curing catalyst (“2E4MZ” manufactured by Shikoku Chemicals Corporation, 0.1 part), spherical silica (average particle size 0.5 μm, “SOC2” manufactured by Admatechs Corporation Limited, 75 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.82). In the same manner as in Example 1, a sheet-like adhesive film was obtained.

### Example 9

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenolic curing agent [an MEK solution (75 parts, solid content 50%) of a novolac resin having a naphthalene structure (“SN-395” manufactured by Tohto Kasei Co., Ltd., phenolic hydroxyl group equivalent weight 107, average hydroxyl group content ratio 2/3˜4/5)], a curing catalyst (“2E4MZ” manufactured by Shikoku Chemicals Corporation, 0.1 part), spherical silica (average particle size 0.5 μm, “SOC2” manufactured by Admatechs Corporation Limited, 60 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.82). In the same manner as in Example 1, a sheet-like adhesive film was obtained.

### Comparative Example 1

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenolic curing agent (50 parts, novolac resin (“LA7052” manufactured by Dainippon Ink and Chemicals Incorporated, MEK solution having a solid content of 60 mass %, phenolic hydroxyl group equivalent amount of solid product 120, average hydroxyl group content ratio 1/1, represented by the following formula (8))), a curing catalyst (“2E4MZ” manufactured by Shikoku Chemicals Corporation, 0.1 part), spherical silica (average particle size 0.5 μm, “SOC2” manufactured by Admatechs Corporation Limited, 55 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.76). In the same manner as in Example 1, a sheet-like adhesive film was obtained.

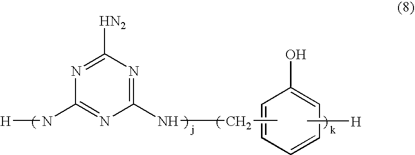

### Comparative Example 2

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenolic curing agent (45 parts, novolac resin (“TD2090-60M” manufactured by Dainippon Ink and Chemicals Incorporated, MEK solution having a solid content of 60 mass %, phenolic hydroxyl group equivalent amount of solid product 105, average hydroxyl group content ratio 1/1, represented by the following formula (9))), a curing catalyst (“2E4MZ” manufactured by Shikoku Chemicals Corporation, 0.1 part), spherical silica (average particle size 0.5 μm, “SOC2” manufactured by Admatechs Corporation Limited, 55 parts), and the polyvinyl butyral resin solution (35 parts) mentioned in Example 1, and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.79). In the same manner as in Example 1, a sheet-like adhesive film was obtained.

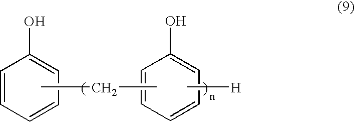

### Comparative Example 3

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenolic curing agent (MEK solution having a solid content of 50%, 110 parts, of novolac resin having a naphthalene structure (“SN-485” manufactured by Tohto Kasei Co., Ltd., phenolic hydroxyl group equivalent amount 215, average hydroxyl group content ratio 1/3˜2/5)), a curing catalyst (“2E4MZ” manufactured by Shikoku Chemicals Corporation, 0.1 part), and spherical silica (average particle size 0.5 μm, “SOC2” manufactured by Admatechs Corporation Limited, 70 parts), and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.79). In the same manner as in Example 1, a sheet-like adhesive film was obtained.

### Comparative Example 4

Liquid bisphenol A epoxy resin (epoxy equivalent weight 180, “Epikote828EL” manufactured by Japan Epoxy Resins Co., Ltd., 28 parts) and naphthalene tetrafunctional epoxy resin (epoxy equivalent weight 163, “EXA-4700” manufactured by Dainippon Ink and Chemicals Incorporated, 28 parts) were dissolved in MEK (15 parts) and cyclohexanone (15 parts) by stirring with heating. Thereto were added a phenoxy resin varnish (mixed solution of cyclohexanone containing a 30 mass % of a nonvolatile component and MEK, “YL6954BH30” manufactured by Japan Epoxy Resins Co., Ltd., glass transition temperature 130° C., 20 parts), a phenolic curing agent (MEK solution having a solid content of 50%, 110 parts, of novolac resin having a naphthalene structure (“SN-485” manufactured by Tohto Kasei Co., Ltd., phenolic hydroxyl group equivalent amount 215, average hydroxyl group content ratio 1/3˜2/5)), a curing catalyst (“2E4MZ” manufactured by Shikoku Chemicals Corporation, 0.1 part), and spherical silica (average particle size 0.5 μm, “SOC2” manufactured by Admatechs Corporation Limited, 70 parts), and the mixture was uniformly dispersed in a high-speed rotor mixer to give resin varnish (inorganic filler content relative to nonvolatile component of resin varnish was 38 mass %, total number of phenolic hydroxyl group/total number of epoxy group was 0.79). In the same manner as in Example 1, a sheet-like adhesive film was obtained.

### Example 10

Production of Multi-Layer Printed Wiring Board:

(1) Preparation of Circuit Substrate:

A circuit pattern is formed by etching on both surfaces of a glass cloth substrate epoxy resin both surface copper-plated laminate board (copper foil thickness 18 μm, substrate thickness 0.8 mm, R5715ES manufactured by Matsushita Electric Works, Ltd.), and the copper surface is roughened by immersion in CZ8100 manufactured by MEC Co., Ltd. to give a circuit substrate.

(2) Lamination of Adhesive Film:

The adhesive films prepared in Examples 1 to 8 are laminated on both surfaces of the circuit substrate using a batch vacuum pressurization laminator MVLP-500 (manufactured by Meiki Co., Ltd., trade name). Lamination comprises depressurizing for 30 seconds to reduce the pressure to not more than 1 3hPa, and pressing for 30 seconds at 0.74 MPa.

(3) Curing of Resin Composition:

The PET film is delaminated from the laminated adhesive film, and the resin composition is cured under the curing conditions of 180° C. for 30 minutes.

(4) Via Hole Formation:

By processing using a CO2-laser processing machine (YB-HCS03T04) manufactured by Matsushita Welding Systems Co., Ltd. under the conditions of frequency 1000 Hz, pulse width 13 μsec, shot number 3, a via hole having a diameter of 60 μm is formed on the surface of the insulating layer.

(5) Roughening Treatment:

The circuit substrate is immersed in a swelling solution, Swelling Dip Securiganth P containing diethyleneglycol monobutylether of Atotech Japan K.K. at 60° C. for 5 minutes. Then, the substrate is immersed in a roughening solution, concentrate compact P (aqueous solution of KMnO₄:60 g/L, NaOH:40 g/L) of Atotech Japan K.K. at 80° C. for 20 minutes. Lastly, the substrate is immersed in a neutralizing solution, Reduction solution Securiganth P of Atotech Japan K.K. at 40° C. for 5 minutes.

(6) Plating and Circuit Formation by Semi-Additive Method:

To form a circuit on the surface of the insulating layer, a circuit substrate is immersed in an electroless plating solution containing PdCl₂, and then immersed in an electroless copper plating solution. After an annealing treatment by heating at 150° C. for 30 minutes, an etching resist is formed and, after formation of a pattern by etching, copper sulfate electroplating is performed. The etching resist is removed, flash etching is further performed, and an anneal treatment is applied at 180° C. for 60 minutes to form an about 25 μm-thick circuit on the surface of the insulating layer, whereby a multi-layer printed wiring board is obtained.

Preparation of sample for measurement of peel strength and Ra value:

(1) Surface Treatment of Laminate Board:

Both surfaces of a glass cloth substrate epoxy resin both surface copper-plated laminate board [copper foil thickness 18 μm, substrate thickness 0.8 mm, RS715ES manufactured by Matsushita Electric Works, Ltd.] were immersed in CZ8100 manufactured by MEC Co., Ltd. for the roughening treatment of the copper surface.

(2) Lamination of Adhesive Film:

The adhesive films prepared in Examples and Comparative Example were laminated on both surfaces of the laminate board using a batch vacuum pressurization laminator MVLP-500 (manufactured by Meiki Co., Ltd., trade name). Lamination comprised depressurizing for 30 seconds to reduce the pressure to not more than 13 hPa, and pressing for 30 sec at 0.74 MPa.

(3) Curing of Resin Composition:

The PET film was delaminated from the laminated adhesive film, and the resin composition was cured under the curing conditions of 180° C. for 30 minutes.

(4) Roughening Treatment:

The laminate board was immersed in a swelling solution, Swelling Dip Securiganth P containing diethyleneglycol monobutylether of Atotech Japan K.K. Then, the board was immersed in a roughening solution, concentrate compact P (aqueous solution of KMnO₄:60 g/L, NaOH:40 g/L) of Atotech Japan K.K. Lastly, the board was immersed in a neutralizing solution, Reduction solution Securiganth P of Atotech Japan K.K. at 40° C. for 5 minutes.

Roughening condition 1: The board was immersed in a swelling solution at 80° C. for 5 minutes and in a roughening solution at 80° C. for 15 minutes.

Roughening condition 2: The board was immersed in a swelling solution at 60° C. for 5 minutes and in a roughening solution at 80° C. for 20 minutes.

The laminate board after the roughening treatment was measured for the surface roughness (Ra value).

(5) Plating by Semi-Additive Method:

To form a circuit on the surface of the insulating layer, the laminate board was immersed in an electroless plating solution containing PdCl₂, and then immersed in an electroless copper plating solution. After an annealing treatment by heating at 150° C. for 30 minutes, an etching resist was formed and, after formation of a pattern by etching, copper sulfate electroplating was performed to give a 25±10 μm thick conductive layer. Then, an anneal treatment was performed at 180° C. for 60 minutes. The peel strength of the plated copper of the laminate board was measured.

Peel Strength of Plated Conductive Layer:

The conductive layer of the laminate board was partly cut in a width of 10 mm and a length of 100 mm, one end thereof was peeled off, grabbed with a clipping tool, and the load necessary for peeling off by 35 mm of the layer in the perpendicular direction at room temperature at a rate of 50 mm/minute was measured.

Surface Roughness (Ra value) after Roughening:

Using a non-contact type surface roughness meter (WYKO NT3300 manufactured by Veeco Instruments), the Ra value was determined. The Ra value is an average value of the height calculated over the whole measurement area, which is concretely an arithmetic average of the measures of the absolute value of height that changes within the measurement areas from the average surface line, and can be represented by the following formula (1) wherein M and N show the number of data in each direction of the array. For the measurement, average roughness of 10 positions was determined.

$$Ra = \frac{1}{MN} {\sum }_{k = 1}^{M} {\sum }_{j = 1}^{N} \vert {Z}_{jk} \vert \qquad ( 1 )$$

The results of the peel strength and surface roughness (Ra value) after roughening of the plated conductive layer of the evaluation samples using the adhesive films obtained in Examples and Comparative Examples are shown in the following Tables 1 to 3. hi the Tables, A1 and A2 show an epoxy resin, C shows polyvinyl acetal, B shows a phenolic curing agent, PR shows a phenoxy resin, SiO₂ shows silica, and CA shows a curing accelerator. As is clear from the Tables, the evaluation samples of Examples using the phenolic curing agent defined by the present invention showed low roughness and high peel strength. In contrast, Comparative Examples using different phenolic curing agents instead of the phenolic curing agent defined by the present invention showed comparatively high peel strength but showed high roughness, and they are not suitable for ultrafine wiring formation. In addition, it is appreciated that Comparative Example 3 free of polyvinyl acetal and Comparative Example 4 containing a phenoxy resin instead of polyvinyl acetal showed low roughness and low peel strength.

TABLE 1

| | | solid | content (parts by mass) | | | | | |
|---|---|---|---|---|---|---|---|---|
| components added | | content (%) | Ex. 1 | Ex. 2 | Ex. 3 | Ex. 4 | Ex. 5 | Ex. 6 |
| 828EL | A1 | 100 | 28 | 28 | 28 | 28 | 28 | 28 |
| EXA4700 | A2 | 100 | 28 | 28 | 28 | 28 | 28 | 28 |
| KS-5Z 15TE | C | 15 | 35 | 35 | 35 | 35 | 35 | 35 |
| LA-7052 | B | 60 | | | | | | |
| TD2090 | B | 60 | | | | | | |
| CBN | B | 50 | | 75 | | | | |
| SN485 | B | 50 | 110 | | | | | |
| GPH-103 | B | 50 | | | | | 120 | |
| NHN | B | 50 | | | | | | 75 |
| YLH1027 | B | 60 | | | | 65 | | |
| YLH1110L | B | 60 | | | 75 | | | |
| YL6954BH30 | PR | 30 | | | | | | |
| SOC2 | SiO2 | 100 | 70 | 60 | 60 | 60 | 75 | 60 |
| 2E4MZ | CA | 100 | 0.1 | 0.1 | 0.1 | 0.1 | 0.1 | 0.1 |

| | | | | | | |
|---|---|---|---|---|---|---|
| peel strength (kgf/cm) | 0.7 | 0.7 | 0.7 | 0.7 | 0.7 | 0.7 |
| roughening conditions 1 | | | | | | |
| peel strength (kgf/cm) | 0.7 | 0.7 | 0.8 | 0.7 | 0.8 | 0.7 |
| roughening conditions 2 | | | | | | |
| surface roughness (nm) | 380 | 320 | 390 | 380 | 390 | 300 |
| roughening conditions 1 | | | | | | |
| surface roughness (nm) | 220 | 250 | 300 | 300 | 200 | 250 |
| roughening conditions 2 | | | | | | |

TABLE 2

| | | | content (parts by mass) | | | | | |
|---|---|---|---|---|---|---|---|---|
| | | solid | | | Comp. | Comp. | Comp. | Comp. |
| components added | | content (%) | Ex. 7 | Ex. 8 | Ex. 1 | Ex. 2 | Ex. 3 | Ex. 4 |
| 828EL | A1 | 100 | 28 | 28 | 28 | 28 | 28 | 28 |
| EXA4700 | A2 | 100 | 28 | 28 | 28 | 28 | 28 | 28 |
| KS-5Z 15TE | C | 15 | 35 | 35 | 35 | 35 | | 35 |
| LA-7052 | B | 60 | | | 50 | | | |
| TD2090 | B | 60 | | | | 45 | | |
| CBN | B | 50 | | 75 | | | | |
| SN485 | B | 50 | 110 | | | | 110 | 110 |
| GPH-103 | B | 50 | | | | | | |
| NHN | B | 50 | | | | | | |
| YLH1027 | B | 60 | | | | | | |
| YLH1110L | B | 60 | | | | | | |
| YL6954BH30 | PR | 30 | 15 | 15 | | | | 20 |
| SOC2 | SiO2 | 100 | 75 | 75 | 55 | 55 | 75 | 70 |
| 2E4MZ | CA | 100 | 0.1 | 0.1 | 0.1 | 0.1 | 0.1 | 0.1 |

| | | | | | | |
|---|---|---|---|---|---|---|
| peel strength (kgf/cm) | 0.7 | 0.7 | 0.7 | 0.5 | 0.3 | 0.4 |
| roughening conditions 1 | | | | | | |
| peel strength (kgf/cm) | 0.8 | 0.8 | 0.8 | 0.6 | 0.3 | 0.3 |
| roughening conditions 2 | | | | | | |
| surface roughness (nm) | 350 | 300 | 700 | 700 | 200 | 200 |
| roughening conditions 1 | | | | | | |
| surface roughness (nm) | 200 | 210 | 600 | 600 | 150 | 150 |
| roughening conditions 2 | | | | | | |

TABLE 3

| | | |
|---|---|---|
| | solid | content (parts |
| | content | by mass) |
| components added | (%) | Ex. 9 |

| | | | |
|---|---|---|---|
| 828EL | A1 | 100 | 28 |
| EXA4700 | A2 | 100 | 28 |
| KS-5Z 15TE | C | 15 | 35 |
| SN395 | B | 50 | 55 |
| SOC2 | SiO2 | 100 | 60 |
| 2E4MZ | CA | 100 | 0.1 |

| | |
|---|---|
| peel strength (kgf/cm) roughening | 0.7 |
| conditions 1 | |
| peel strength (kgf/cm) roughening | 0.7 |
| conditions 2 | |
| surface roughness (nm) roughening | 320 |
| conditions 1 | |
| surface roughness (nm) roughening | 240 |
| conditions 2 | |

The resin composition of the present invention, and the adhesive film and the prepreg prepared from the resin composition are preferably used as an interlayer insulating material of a multi-layer printed wiring board, particularly a multi-layer printed wiring board produced by a build-up method.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An epoxy resin composition, comprising:
(A) at least one epoxy resin having two or more epoxy groups in one molecule;
(B) at least one phenolic curing agent having an average content ratio of hydroxyl group in one molecule (P) of 0<P<1; and
(C) at least one polyvinyl acetal resin,
wherein the average content ratio of hydroxyl group in one molecule (P) is an average value of H/B, where H is the total number of hydroxyl groups and B is the total number of benzene rings.

2. The epoxy resin composition of claim 1, wherein said phenolic curing agent (B) is a phenolic curing agent represented by formula (1) or formula (2): 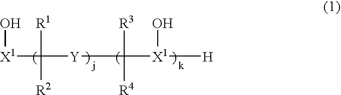
wherein R¹ to R⁴ are each independently hydrogen atom or an alkyl group; X¹ is a benzene ring, hydroxyl benzene ring, naphthalene ring or hydroxyl naphthalene ring each of which may be substituted with an alkyl group; Y is a benzene ring, hydroxyl benzene ring or a biphenyl ring each of which may be substituted with an alkyl group; and j and k are each independently number of 1 to 15 as average value, 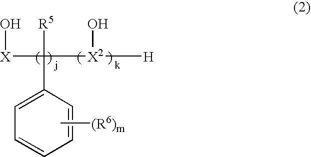
wherein R⁵ is hydrogen atom or an alkyl group; R⁶ is hydrogen atom, an alkyl group, or a thioalkyl group; X² is a benzene ring or naphthalene ring each of which may be substituted with an alkyl group; Y is a benzene ring, hydroxyl benzene ring or a biphenyl ring each of which may be substituted with an alkyl group; j and k are each independently number of 1 to 15 as average value; and m is an integer of 1 to 5.

3. The epoxy resin composition of claim 1, wherein said phenolic curing agent (B) is a phenolic curing agent represented by formula (1′) or (2′): 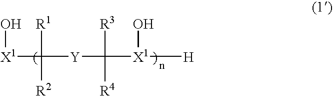
wherein R1 to R4 are each independently a hydrogen atom or an alkyl group, X1 is a benzene ring, hydroxyl benzene ring, naphthalene ring or hydroxyl naphthalene ring, each optionally substituted by an alkyl group, Y is a benzene ring, a hydroxybenzene ring or a biphenyl ring, each optionally substituted by an alkyl group, and n is a number of 1 to 15 as average value, 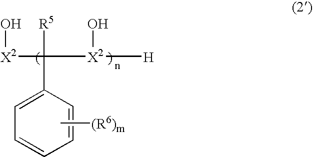
wherein R5 is a hydrogen atom or an alkyl group, R6 is a hydrogen atom, an alkyl group or a thioalkyl group, X2 is a benzene ring or a naphthalene ring, each optionally substituted by an alkyl group, n is a number of 1 to 15 as average value, and m is an integer of 1 to 5.

4. The epoxy resin composition of claim 1, wherein said phenolic curing agent
(B) is a phenolic curing agent represented by any of formulas (3) to (5): 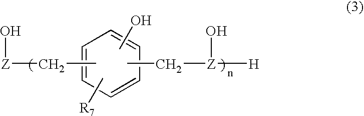
wherein R7 is a hydrogen atom or a methyl group, Z is a naphthalene ring, and n is a number of 1 to 15 as average value; 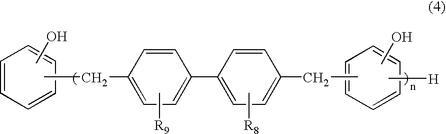
wherein R8 and R9 are each independently a hydrogen atom or a methyl group, and n is a number of 1 to 15 as average value; and 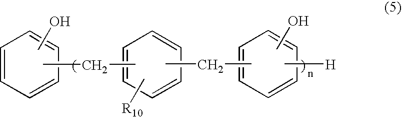
wherein R10 is a hydrogen atom or a methyl group, and n is a number of 1 to 15 as average value.

5. The epoxy resin composition of claim 1, wherein said phenolic curing agent (B) is a phenolic curing agent represented by formula (6): 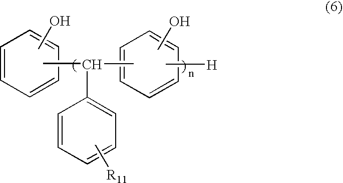
wherein R11 is a hydrogen atom, a methyl group, or a thiomethyl group, and n is a number of 1 to 15 as average value.

6. The epoxy resin composition according to claim 1, wherein the phenol type curing agent of the component (B) is a phenol type curing agent represented by formula (7): 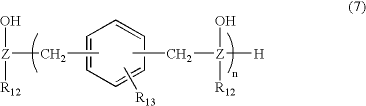
wherein R12 is hydrogen atom, a methyl group or hydroxyl group; R13 is hydrogen atom, a methyl group or hydroxyl group; Z is a naphthalene ring; and n is a number of 1 to 15 as average value.

7. The epoxy resin composition of claim 1, wherein said epoxy resin (A) comprises:
(A1) at least one first epoxy resin (A1) that is an aromatic epoxy resin having two or more epoxy groups in one molecule, which is liquid at a temperature of 20° C.; and
(A2) at least one second epoxy resin (A2) that is an aromatic epoxy resin having three or more epoxy groups in one molecule, which is solid at a temperature of 20° C.

8. The epoxy resin composition of claim 7, wherein said second epoxy resin (A2) has an epoxy equivalent weight of not more than 230.

9. The epoxy resin composition of claim 7, wherein said second epoxy resin (A2) has an epoxy equivalent weight within the range of 150 to 230.

10. The epoxy resin composition of claim 7, which comprises 0.3 to 2 parts by weight of said second epoxy resin (A2) per 1 part by weight of said first epoxy resin (A1).

11. The epoxy resin composition of claim 1, wherein said polyvinyl acetal resin (C) has a glass transition temperature of not less than 80° C.

12. The epoxy resin composition of claim 1, wherein said epoxy resin composition comprises 10 to 50 parts by mass of the epoxy resin (A) and 2 to 20 parts by mass of the polyvinyl acetal resin (C) per the total 100 parts by mass of the nonvolatile component, and
a ratio of number (E) of epoxy groups and number (P) of phenolic hydroxyl groups of the phenolic curing agent (B), P/E, present in the epoxy resin composition is 0.5 to 1.5.

13. The epoxy resin composition of claim 1, which further comprises:
(D) at least one phenoxy resin.

14. The epoxy resin composition of claim 13, which comprises 1 to 20 parts by mass of a phenoxy resin per 100 parts by mass of nonvolatile components in said epoxy resin composition.

15. The epoxy resin composition of claim 1, which further comprises:
(D′) at least one inorganic filler.

16. The epoxy resin composition of claim 15, which comprises 10 to 75 parts by mass of an inorganic filler per 100 parts by mass of nonvolatile components in the epoxy resin composition.

17. An adhesive film, which comprises a layer comprising an epoxy resin composition of claim 1, which is formed on a support film.

18. A prepreg, which comprises a sheet-like reinforcement substrate made of a fiber, which is impregnated with an epoxy resin composition of claim 1.

19. A multi-layer printed wiring board, comprising an insulating layer obtained by curing an epoxy resin composition of claim 1.

20. A method of making a multi-layer printed wiring board, which comprises:
(1) heat curing an epoxy resin composition of claim 1 on an inner layer circuit substrate, to obtain an insulating layer;
(2) roughening a surface of said insulating layer, to obtain a roughened surface; and
(3) forming a conductive layer on the roughened surface by copper plating.

21. A method of making a multi-layer printed wiring board, which comprises:
(1) forming an insulating layer on an inner layer circuit substrate by laminating the adhesive film of claim 17 on the inner layer circuit substrate, heat curing the epoxy resin composition with or without delamination of the above-mentioned support film, after which delaminating the support film when the support film is present;
(2) roughening a surface of said insulating layer, to obtain a roughened surface; and
(3) forming a conductive layer on the roughened surface by copper plating.

22. A method of making a multi-layer printed wiring board, which comprises:
(1) forming an insulating layer on an inner layer circuit substrate by laminating the prepreg of claim 18 on the inner layer circuit substrate and heat curing the epoxy resin composition;
(2) roughening a surface of said insulating layer, to obtain a roughened surface; and
(3) forming a conductive layer on the roughened surface by copper plating.

23. The method of claim 20, wherein said roughening comprises exposing said surface of said insulating layer to an oxidant.

24. The method of claim 21, wherein said roughening comprises exposing said surface of said insulating layer to an oxidant.

25. The method of claim 22, wherein said roughening comprises exposing said surface of said insulating layer to an oxidant.

26. The method of claim 20, wherein said roughening comprises exposing said surface of said insulating layer to an alkaline permanganate solution.

27. The method of claim 21, wherein said roughening comprises exposing said surface of said insulating layer to an alkaline permanganate solution.

28. The method of claim 22, wherein said roughening comprises exposing said surface of said insulating layer to an alkaline permanganate solution.

29. The method of claim 20, wherein said roughened surface has a roughness in an Ra value of not more than 0.5 μm and said conductive layer has a peel strength of not less than 0.6 kgf/cm.

30. The method of claim 21, wherein said roughened surface has a roughness in an Ra value of not more than 0.5 μm and said conductive layer has a peel strength of not less than 0.6 kgf/cm.

31. The method of claim 22, wherein said roughened surface has a roughness in an Ra value of not more than 0.5 μm and said conductive layer has a peel strength of not less than 0.6 kgf/cm.
